(19) United States
(12) Patent Application Publication
(10) Pub. No.: US 2009/0171205 A1
(43) Pub. Date: Jul. 2, 2009

(54) METHOD AND SYSTEM FOR LOCATING BLOOD VESSELS

(75) Inventors: Aleksey Kharin, Eindhoven (NL); Sieglinde Neerken, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/159,636

(22) PCT Filed: Dec. 28, 2006
(86) PCT No.: PCT/IB06/55049

§ 371 Date: Dec. 22, 2008

(30) Foreign Application Priority Data
Jan. 3, 2006 (EP) 06300005.3

(51) Int. Cl. A61B 8/00; A61B 6/00

(52) U.S. Cl. 600/443; 600/476

(57) ABSTRACT

A method and system for detecting blood vessels and precisely determining parameters in respect thereof, such as geometry, depth and diameter. Optical imaging, such as IR imaging, is used to obtain a general overview (16) of the blood vessel pattern in a region of interest from which a target vessel can be defined and its lateral position determined. Ultrasound imaging is then employed to precisely measure parameters, such as depth and diameter, in respect of the target vessel. The resultant system is of relatively low cost, and relatively simple to implement and use.

This invention relates generally to a method and system for locating blood vessels, for example, for use by a practitioner to precisely detect the position of a blood vessel into which a surgical needle is to be inserted, either manually or in a fully automated robotic system for performing invasive medical procedures.

There are many circumstances in which a practitioner may be required to insert a surgical needle into a blood vessel, for example, for the purpose of injecting a substance into the blood, withdrawing blood, or inserting a catheter. Insertion of a needle into a blood vessel is often difficult to achieve due to problems in finding a blood vessel and then precisely positioning the needle in the selected blood vessel.

Ultrasound imaging is a well-known technique for the detection and localization of boundaries between two media with different acoustical impedance. Short bursts of ultrasound radiation are applied to the region of interest and the amplitude and arrival time of the reflected and backscattered signals are measured so as to map the boundaries between two media with different acoustical impedance and acoustical attenuation. This technique is widely used in fields such as materials and medicine.

Thus, ultrasound imaging can be used to accurately determine the depth, diameter and shape of a blood vessel. However, in most cases, two-dimensional data is obtained using this technique and, in order to obtain a three-dimensional overview of the course of the vessel, the ultrasound probe needs to be scanned. Furthermore, it can be difficult to distinguish the vessel from the surrounding tissue, and image reconstruction and pattern recognition algorithms are required to be utilised in order to derive the blood vessel course and position parameters, which further increases the cost and complexity of the system.

It is therefore an object of the invention to provide a method and system for determining parameters of a blood vessel, such as location, depth and/or diameter, which is more accurate and less costly and complex than prior art arrangements.

In accordance with the present invention, there is provided a method for determining one or more parameters of a blood vessel, the method comprising the steps of illuminating a region of interest of a subject with electromagnetic radiation, receiving electromagnetic radiation reflected from or transmitted through said subject and generating image data representative of the intensity distribution of said received electromagnetic radiation, identifying, from said image data, a target area within said region of interest, positioning an ultrasonic transducer relative to said target area and applying ultrasonic radiation at said target area, receiving ultrasonic radiation reflected and/or backscattered from said target area of said subject, measuring the amplitude and/or arrival time of said received ultrasonic radiation so as to identify boundaries in respect of a blood vessel within said target area between the wall of said blood vessel and its surroundings, and determining therefrom at least one dimension of said blood vessel.

Also in accordance with the present invention, there is provided a system for determining one or more parameters of a blood vessel, the system comprising means for illuminating a region of interest of a subject with electromagnetic radiation, means for receiving electromagnetic radiation reflected from or transmitted through said subject and generating image data representative of the intensity distribution of said received electromagnetic radiation, means for enabling identification, from said image data, of a target area within said region of interest and enabling positioning of an ultrasonic transducer relative to said target area, means for applying ultrasonic radiation at said target area, means for receiving ultrasonic radiation reflected and/or backscattered from said target area of said subject, means for measuring the amplitude and/or arrival time of said received ultrasonic radiation so as to identify boundaries in respect of a blood vessel within said target area between the wall of said blood vessel and its surroundings, and means for determining therefrom at least one of a dimension or depth of said blood vessel.

Thus, the present invention achieves the above-mentioned object by first enabling an overview of the vessel pattern in the region of interest, so as to enable a target blood vessel, and its lateral position within the region of interest, to be identified. Only then is an ultrasonic imaging technique used to “zoom” at the selected lateral position to determine the depth and/or diameter of the target blood vessel.

In a preferred embodiment, the electromagnetic radiation comprises optical radiation and, more preferably, infrared or near-infrared radiation.

The image data in respect of the region of interest is preferably displayed (to provide a practitioner with an overview of the vessel pattern in the region of interest) and selection of the target area, and positioning of the ultrasonic transducer relative thereto, may be performed manually. However, shape recognition techniques may alternatively be employed to determine the target area automatically and motive means may be provided to automatically position the ultrasonic transducer relative to the selected target area, whether selected manually or automatically.

These and other aspects of the present invention will be apparent from, and elucidated with reference to, the embodiments described herein.

Embodiments of the present invention will now be described by way of examples only and with reference to the accompanying drawings, in which:

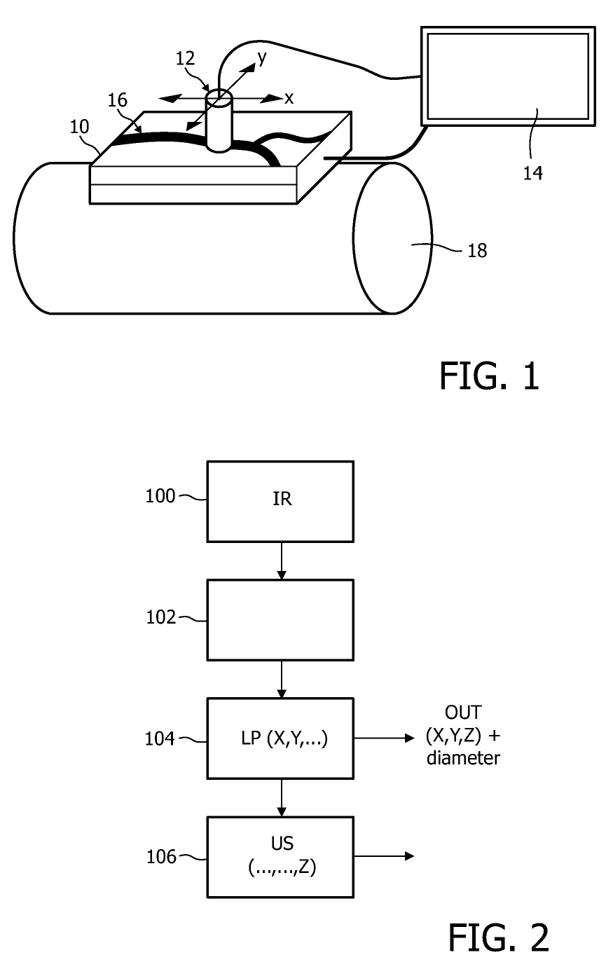

FIG. 1 is a schematic diagram illustrating the principal components of a system according to an exemplary embodiment of the present invention;

FIG. 2 is a schematic flow diagram illustrating the principal steps of a method according to an exemplary embodiment of the present invention;

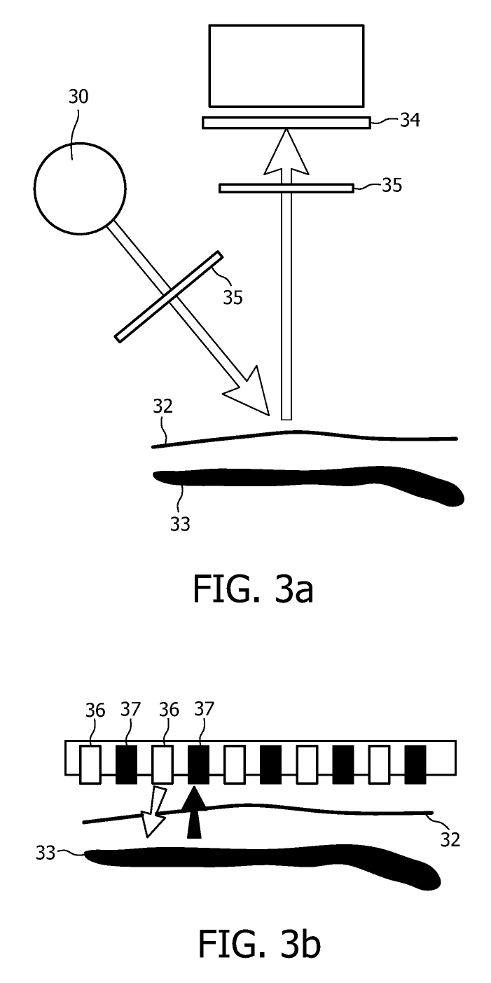

FIG. 3a is a schematic illustration of an optical imaging system capable of taking an image of an integrated object remotely; and

FIG. 3b is a schematic illustration of an optical imaging system capable of taking an image of an investigated object very close or in direct contact therewith.

Referring to FIG. 1 of the drawings, a system according to an exemplary embodiment of the present invention comprises an infrared imaging unit 10 (most preferably the one shown in FIG. 3b) and an acoustic transducer 12 connected to, or provided integrally, the IR imaging unit 10. The acoustic transducer comprises of means to convert an electrical signal into a mechanical vibration and vice versa. The IR imaging unit 10 and the acoustic transducer (12) are connected to a signal processing module 14, which may be provided in the form of a PC or the like.

Referring additionally to FIG. 2 of the drawings, the IR imaging unit 10 generates (at step 100) an IR image 16 of a region of interest of the subject 18 under investigation by illuminating the region of interest with infrared radiation, receiving backscattered radiation from the region of interest and generating image data representative of the intensity distribution of the backscattered radiation. The image data is then transferred to the signal processing module 14, enhanced using known image processing techniques, vessel map is derived (step 102), target vessel is chosen and the two-dimensional co-ordinates of the target blood vessels is finally derived (step 104) from the resultant image of the region of interest. The depth and size of the vessel is detected (step 106) using acoustical transducer (12) and signal processing module (14). Output data of the method is the sum of lateral position of chosen (either manually or automatically) target vessel (step 104) and depth and diameter of vessel (step 106).

Referring additionally to FIG. 3a of the drawings, the investigated object is exposed to radiation emitted by radiation source unit 30. Two dimensional map of light distribution on the surface of the investigated body 32 is measured by light detector e.g. camera 31. Depending on particular implementation the wavelength filter 34 can be used to reduce detector noise. Depending on requirements the use of polirizers shifted for 90 degree (35) can increase visibility of structures lying under surface of investigated object (33) by reduction of amount of light reflected from surface of investigated object.

Referring additionally to FIG. 3b of the drawings, the investigated object is exposed to radiation emitted by matrix of light sources (36). Two dimensional map of light distribution on the surface of the investigated body (32) is measured by matrix of light detectors (37) placed directly very close to investigated body.

Depending on the construction of the optical imaging system, means (10) may comprise of means (31) or (36) for limitation of the investigated object and means (30) or (37) to map the light distribution on the surface of the investigated object.

Optical imaging techniques, such as infrared imaging are well known and are based on illumination of the investigated subject and the detection of photons reflecting from or travelling through the subject. This technique enables the optical properties of the subject under investigation to be mapped, and has key advantages that the presence of a blood vessel and its lateral position can be relatively easily determined in real-time and at relatively low cost. The precise depth and dimensions such as diameter of the vessel, on the other hand, are more difficult to derive using this technique. There are some known methods using optical imaging that can provide an approximate depth reconstruction only, and these are in any event expensive, time consuming and difficult to implement.

Instead, therefore, in accordance with the invention, once an overview of the blood vessel pattern has been obtained using IR imaging, a blood vessel into which a surgical needle is (potentially) to be inserted is then selected (at step 102) using the two-dimensional representation of the geometry of the blood vessels, and its lateral position (LP) obtained (at step 104). Next, the acoustic transducer 12 is positioned (either manually or automatically) at the location of the selected vessel and the vessel depth and/or diameter, for example, may be determined using known ultrasound imaging and/or measurement techniques, and output in a required format.

Thus, a general overview of the region of interest can be obtained using IR imaging, so as to provide an overview of the blood vessel pattern. This allows a target vessel to be defined and its lateral position determined. “Zooming” in the selected lateral position with ultrasonic imaging techniques then allows the depth and dimensions of the target vessel to be accurately determined. Ultrasound equipment required for this application can be less complex and thus cheaper than conventional high-end ultrasound scanners.

There are many circumstances in which it is required to accurately locate a blood vessel for needle insertion and in which the present invention would be suitable for use. Key advantages include highly accurate data relating to location and parameters of a blood vessel, at relatively low cost.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. A method for determining one or more parameters of a blood vessel, the method comprising the steps of illuminating a region of interest of a subject (18) with electromagnetic radiation, receiving electromagnetic radiation reflected from or transmitted through said subject (18) and generating (100) image data (16) representative of the intensity distribution of said received electromagnetic radiation, identifying (102) from said image data (16), a target area within said region of interest, positioning an ultrasonic transducer (12) relative to said target area and applying (106) ultrasonic radiation at said target area, receiving ultrasonic radiation reflected and/or backscattered from said target area of said subject (18), measuring the amplitude and/or arrival time of said received ultrasonic radiation so as to identify boundaries in respect of a blood vessel within said target area between the wall of said blood vessel and its surroundings, and determining therefrom at least one dimension of said blood vessel.

2. A method according to claim 1, wherein said electromagnetic radiation comprises optical radiation.

3. A method according to claim 2, wherein said optical radiation comprises infrared or near-infrared radiation.

4. A method according to claim 1, wherein said image data (16) in respect of said region of interest is displayed and selection of the target area, and positioning of the ultrasonic transducer (12) relative thereto is performed using said displayed image data.

5. A method according to claim 1, wherein positioning of said ultrasonic transducer (12) relative to said target area is performed manually.

6. A method according to claim 1, wherein positioning of said ultrasonic transducer (12) relative to said target area is performed automatically by motive means.

7. A system for determining one or more parameters of a blood vessel, the system comprising means (10) for illuminating a region of interest of a subject (16) with electromagnetic radiation, means for receiving electromagnetic radiation reflected from or transmitted through said subject and generating image data (16) representative of the intensity distribution of said received electromagnetic radiation, means (14) for enabling identification, from said image data, of a target area within said region of interest and enabling positioning of an ultrasonic transducer (12) relative to said target area, means for applying ultrasonic radiation at said target area, means for receiving ultrasonic radiation reflected and/or backscattered from said target area of said subject (18), means (14) for measuring the amplitude and/or arrival time of said received ultrasonic radiation so as to identify boundaries in respect of a blood vessel within said target area between the wall of said blood vessel and its surrounding, and means (14) for determining therefrom at least one of a dimension or depth of said blood vessel.
